(19) United States
(12) Patent Application Publication
(10) Pub. No.: US 2025/0166741 A1
(43) Pub. Date: May 22, 2025

(54) MACHINE LEARNING (ML)-ACCELERATED FIRST-PRINCIPLES PREDICTION METHOD FOR HYDRATION STRUCTURE OF ACID RADICAL ANION

(71) Applicant: Central South University, Changsha (CN)

(72) Inventors: Chenyang ZHANG, Changsha (CN); Mingjun HAN, Changsha (CN); Dong FAN, Changsha (CN); Wei SUN, Changsha (CN); Jie LI, Changsha (CN); Yong PEI, Changsha (CN); Guixiang ZENG, Changsha (CN); Shangyong LIN, Changsha (CN); Hongliang ZHANG, Changsha (CN); Rong WANG, Changsha (CN)

(73) Assignee: Central South University, Changsha (CN)

(21) Appl. No.: 18/827,929

(22) Filed: Sep. 9, 2024

(30) Foreign Application Priority Data
Nov. 20, 2023 (CN) 202311547565 .7

(51) Int. Cl. G16C 20/70; G16C 20/50

(52) U.S. Cl. CPC G16C 20/70; G16C 20/50

(57) ABSTRACT

A machine learning (ML)-accelerated first-principles prediction method for a hydration structure of an acid radical anion is provided. The prediction method includes the following steps: S1: constructing and optimizing an anion hydration structure M_mH₂O; S2: perturbing the optimized anion hydration structure to generate a training dataset; S3: conducting a ML force field training on the training dataset to establish ML models; S4: conducting a molecular dynamics simulation on the ML models, and identifying atomic structures with a force deviation within a preset range as candidate configurations; S5: merging a validated candidate configuration into a training set for a subsequent iteration to further refine and train the ML model until the model converges, thereby generating an accurate deep potential (DP) model; and S6: conducting a ML-accelerated deep potential molecular dynamics simulation on the DP model to ultimately acquire the hydration structure of the acid radical anion.

### CROSS REFERENCE TO THE RELATED APPLICATIONS

This application is based upon and claims priority to Chinese Patent Application No. 202311547565.7, filed on Nov. 20, 2023, the entire contents of which are incorporated herein by reference.

### TECHNICAL FIELD

The present disclosure relates to the prediction of a hydration structure of an acid radical anion, and in particular to a machine learning (ML)-accelerated first-principles prediction method for a hydration structure of an acid radical anion.

### BACKGROUND

The hydration structure around ions has a significant impact on the electron transfer and reaction rate of chemical reaction processes. Hydration can change the driving force of chemical reactions, making it a key factor to consider in selective actions. Currently, there are reports and studies on the hydration structures of metal cations, such as Pb(II), Cu(II), Fe(II), Fe(III), Zn(II), V(II), V(III), Ca(II), and Mg(II), in aqueous solutions. However, there are few studies on the hydration structures of acid radical anions formed by metal cations and oxygen or sulfur, such as WO₄²⁻ and MoS₄²⁻.

In order to more accurately simulate the real environment of ion hydration, it is necessary to consider the large number of water molecules around the ions, which makes it impossible for precise quantitative measurements in traditional experiments. Quantum chemistry theory calculations can simulate and predict complex chemical reactions through high-speed computers and precise quantum mechanics algorithms. At present, ab initio molecular dynamics (AIMD) simulations have basically solved the problem of “inaccuracy”, but the problems of “slow calculation” and “high cost” still exist. For example, the simulation of a WO₄²⁻_ 100H₂O_2Na system includes 4,000 steps, taking a total of 2 ps, 0.5 fs of time for each step, conducted on a high-performance computing server with a total of 96 cores at 2 nodes. It requires uninterrupted operation for about 12 days, resulting in a computing service fee of approximately 1,400 yuan (0.05 yuan/core hour). Obviously, a 100 ps AIMD simulation requires uninterrupted operation for about 60 days and costs approximately 70,000 yuan. Such slow computing progress and expensive computing costs cannot achieve the cost-effective purpose.

Therefore, it is necessary to establish a systematic research method to accurately and quickly predict the hydration structures of ions, especially acid radical anions such as WO₄²⁻ and MoS₄²⁻.

### SUMMARY

In view of the problems of “slow calculation” and “high cost” in predicting the hydration structures of acid radical anions, the present disclosure provides a machine learning (ML)-accelerated first-principles prediction method for a hydration structure of an acid radical anion. The prediction method greatly improves computational efficiency and reduces computational costs while ensuring computational accuracy.

In order to achieve the above objective, the present disclosure provides a ML-accelerated first-principles prediction method for a hydration structure of an acid radical anion (such as WO₄²⁻, MoS₄²⁻, SO₄²⁻, or CO₃²⁻), including the following steps:
S1: constructing and optimizing an anion hydration structure M_mH₂O;
S2: perturbing the optimized anion hydration structure to generate a training dataset;
S3: conducting a ML force field training on the training dataset to establish ML models;
S4: conducting a molecular dynamics simulation on the ML models, and identifying atomic structures with a force deviation within a preset range as candidate configurations;
S5: merging a validated candidate configuration into a training set for a subsequent iteration to further refine and train the ML model until the model converges, thereby generating an accurate deep potential (DP) model; and
S6: conducting a ML-accelerated molecular dynamics simulation on the DP model to ultimately acquire the hydration structure of the acid radical anion.

The above technical solution achieves a deep potential molecular dynamics (DPMD) simulation on the anion hydration structure, especially achieves the first rapid and accurate prediction for the hydration structure of the acid radical anion. A traditional AIMD simulation method can generally only achieve a 1-10 ps simulation, while the DPMD simulation method can easily achieve a 100-1,000 ps simulation. The present disclosure greatly improves computational efficiency and reduces computational costs while ensuring computational accuracy.

Specifically, in the step S1, a method for constructing the anion hydration structure through Materials Studio software includes: creating a periodic simulation box, placing the acid radical anion M at a center of the box, uniformly and randomly distributing m H₂O around the acid radical anion M, and placing an alkali metal cation at an edge of the box to balance a charge, thereby completing the construction of the initial anion hydration structure M_mH₂O, where m is above 40.

In the above technical solution, careful consideration should be given when selecting the initial arrangement of H₂O and the initial distance between M and H₂O to ensure that the simulated initial state is reasonable.

On the basis of the above technical solution, in order to more accurately describe and construct the hydration structure of the anion M, before constructing the anion hydration structure through first-principles density functional theory (DFT), the prediction method further includes pre-optimizing: preparing a def2-TZVP, def2-SVP, def2-QZVP, def2-TZVPP, cc-pVDZ-PP or aug-cc-pVDZ-PP basis set through an ωB97XD, B3LYP or PBE functional; and pre-optimizing a simple hydration structure M_nH₂O formed by n H₂O in a sternest layer around the anion M to acquire a stern layer hydration structure of the acid radical anion M. The step S1 further includes: uniformly and randomly distributing remaining (m-n) H₂O around M_nH₂O, thereby completing the construction of the initial anion hydration structure M_mH₂O; and optimizing the constructed anion hydration structure through Vienna ab initio simulation package (VASP).

Specifically, in the step S2, the perturbing includes: changing an atomic coordinate position and a size of the simulation box to generate A different perturbed structures, each perturbed structure being in a canonical ensemble (NVT ensemble) at 298.15 K; and recording B frames for each configuration at a time step of 0.5-1.5 fs, and conducting a short-term (step size×frame rate) AIMD simulation to generate a training dataset of A×B frames as basic training data for a DeepMD model, where A is 15-30; and B is 15-25.

Preferably, the time step is 0.5 fs; A is 20; and B is 20.

The perturbation is to appropriately change the atomic coordinate position and the size of the simulation box based on the already optimized structure, in order to further acquire more acceptable structures and generate the training dataset.

Specifically, the step S3 includes: conducting the ML force field training on the training dataset through deep potential generator (DP-GEN) and DeePMD kit software package, where the ML force field training includes 2×10⁵ to 8×10⁵ steps, with 2-5 independent ML models established each time. These models use the same reference dataset but have different initial weight values. Preferably, there are 4×10⁵ training steps and 4 independent ML models.

Specifically, in the step S4, the conducting a molecular dynamics simulation includes: conducting, by a large-scale atomistic/molecular massively parallel simulator (LAMMPS), the molecular dynamics simulation on the anion hydration structure in the NVT ensemble under at least five different temperature conditions; and identifying, during the simulation, atomic structures with a force deviation ranging within 0.11-0.30 eV/Å as candidate configurations, where a maximum of 300 candidate configurations are selected.

The LAMMPS interface is complete and easy to expand, allowing for fast molecular dynamics parallel simulations on force field models trained through ML force field trainings, in order to select candidate configurations within the range of force deviations.

In a preferred implementation of the present disclosure, the prediction method includes: conducting, by the LAMMPS, the molecular dynamics simulation on the anion hydration structure in the NVT ensemble under five different temperature conditions of 250 K, 280 K, 300 K, 320 K, and 350 K.

For the selection of these five temperatures, a temperature gradient is constructed within the range of 250-350 K, taking into account common temperature conditions to ensure a more reasonable hydration structure.

Specifically, the step S5 includes: merging a candidate configuration with an energy and atomic force validated through the DFT into the training set for the subsequent iteration to further refine and train the ML model. For all iterative processes, automation is achieved through the DP-GEN software package until the model converges to acquire an accurate DP model.

Specifically, the step S6 includes: applying a trained ML force field to the LAMMPS, and conducting a 50-1,000 ps ML-accelerated deep potential molecular dynamics (i.e. DPMD) simulation on the DP model to ultimately acquire the hydration structure of the acid radical anion.

The present disclosure adopts the DPMD method, which greatly improves the simulation calculation speed and enables the accuracy of simulation results to be comparable to that of traditional AIMD methods, providing an efficient and accurate ML-accelerated method for first-principles prediction of anion hydration structures. Compared with traditional AIMD methods, in the analysis and validation stage of simulation results, the present disclosure can further validate the accuracy and efficiency of the ML-accelerated method, while exploring the impact of different simulation durations and conditions on simulation results and how to optimize and improve the technical solution to enhance accuracy and efficiency.

With the above technical solution, the present disclosure achieves the following beneficial effects:

The present disclosure implements the construction of the anion hydration structure, perturbation, ML force field training, molecular dynamics simulation, and model convergence. The present disclosure ultimately achieves DPMD simulation on the anion hydration structure, particularly achieving the first rapid and accurate prediction for the hydration structure of the acid radical anion. The traditional AIMD simulation method can generally only achieve a 1-10 ps simulation, while the DPMD simulation method can easily achieve a 100-1,000 ps simulation. The present disclosure greatly improves computational efficiency and reduces computational costs while ensuring computational accuracy.

### BRIEF DESCRIPTION OF THE DRAWINGS

FIG. 1 is a flowchart of first-principles prediction method for a hydration structure of an acid radical anion according to an embodiment of the present disclosure;

FIGS. 2A-2B show initial configurations of molecular dynamics simulations for [MoS₄(H₂O)₆₄]²⁻ and [WO₄(H₂O)₆₄]²⁻ according to Embodiments 1 and 2 of the present disclosure, where FIG. 2A shows the initial configuration of the molecular dynamics simulation for [MoS₄(H₂O)₆₄]²⁻, and FIG. 2B shows the initial configuration of the molecular dynamics simulation for [WO₄(H₂O)₆₄]²⁻;

FIGS. 3A-3B show hydration structures of [MoS₄(H₂O)₆₄]²⁻ and [WO₄(H₂O)₆₄]²⁻ in a 100 ps DPMD simulation according to Embodiments 1 and 2, where FIG. 3A shows the hydration structure of [MoS₄(H₂O)₆₄]²⁻ in the 100 ps DPMD simulation, and FIG. 3B shows the hydration structure of [WO₄(H₂O)₆₄]²⁻ in the 100 ps DPMD simulation; and

FIGS. 4A-4B show hydration structures of SO₄(H₂O)₆₄]²⁻ and [CO₃(H₂O)₆₄]²⁻ in a 100 ps DPMD simulation according to Embodiment 3, where FIG. 4A shows the hydration structure of [SO₄(H₂O)₆₄]²⁻ in the 100 ps DPMD simulation, and FIG. 4B shows the hydration structure of [CO₃(H₂O)₆₄]²⁻ in the 100 ps DPMD simulation.

### DETAILED DESCRIPTION OF THE EMBODIMENTS

The specific implementation of the present disclosure is described in detail below based on embodiments. It should be understood that the specific implementations described herein are merely intended to illustrate and interpret the present disclosure, rather than to limit the present disclosure.

### Embodiment 1: Thiomolybdate Anion Hydration (MoS₄²⁻_64H₂O_2Na)

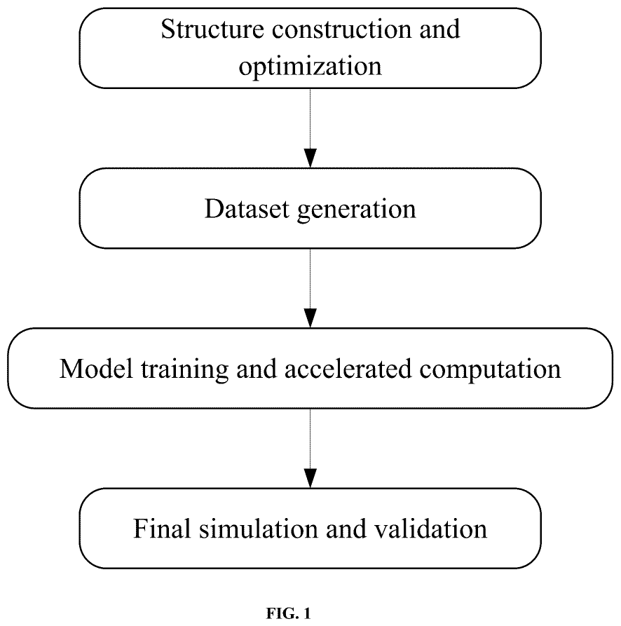

As shown in FIG. 1, a prediction method for thiomolybdate anion hydration (MoS₄²⁻_64H₂O_2Na) is as follows.

### 1. Structure Construction and Optimization

In order to more accurately describe and construct a hydration structure of MoS₄²⁻, based on first-principles DFT, the structure of MoS₄²⁻_6H₂O is pre-optimized through a def2-TZVP basis set prepared by an ωB97XD functional, thereby generating a stern layer hydration structure of MoS₄²⁻.

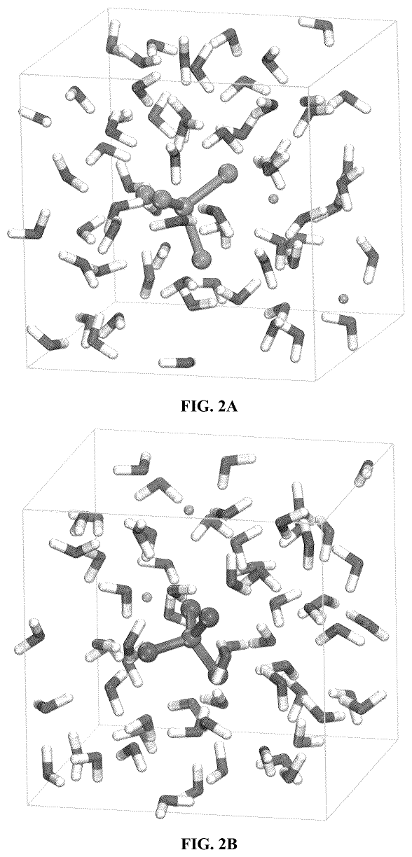

The anion hydration structure is constructed through Materials Studio software. As shown in FIG. 2A, a MoS₄²⁻_64H₂O_2Na model is constructed in a periodic box of 15×15×15 Å. MoS₄²⁻ is located at a center of the box, while the remaining 58 H₂O are uniformly and randomly distributed around MoS₄²⁻_6H₂O, and Na⁺ is placed at an edge of the box to balance a charge. The constructed anion hydration structure MoS₄²⁻_64H₂O_2Na is optimized through VASP.

### 2. Dataset Generation

The optimized MoS₄²⁻_64H₂O_2Na structure is perturbed, including changing an atomic coordinate position and a size of the simulation box to generate 20 different perturbed structures. A short-term AIMD simulation is conducted on the optimized structure MoS₄²⁻_64H₂O_2Na in an NVT ensemble at 298.15 K, with each configuration spanning 20 frames based on a time step of 0.5 fs.

In this way, a training dataset of the hydration structure MoS₄²⁻_64H₂O_2Na is acquired as basic training data for a DeepMD model. It includes 20 perturbed structures, each with 20 frames, totaling 400 frames.

### 3. Model Training and Accelerated Computation

Through DP-GEN and DeePMD kit software package, a ML force fields training is conducted on the training dataset of the hydration structure MoS₄²⁻_64H₂O_2Na.

Firstly, a 4×10⁵-step training process is conducted on the training dataset of MoS₄²⁻_64H₂O_2Na. 4 independent ML models are established for each training session. These models use the same reference dataset but have different initial weight values.

Then, the trained ML model is applied to a molecular dynamics simulation to explore a new anion hydration structure. The molecular dynamics simulation is conducted by a LAMMPS on the anion hydration structure MoS₄²⁻_64H₂O_2Na in the NVT ensemble under five different temperature conditions of 250 K, 280 K, 300 K, 320 K, and 350 K. During the simulation, atomic structures with a force deviation ranging within 0.11-0.30 eV/Å are identified as candidate configurations, and a maximum of 300 candidate configurations are selected.

Finally, a candidate configuration with an energy and atomic force validated through the DFT is merged into the training set for the subsequent iteration to further refine and train the ML model. All iterative processes are automated through the DP-GEN software package until the model converges (i.e., the energy accuracy of the sampled data reaches over 95%) to acquire an accurate DP model. During the validation and testing phase of the model, different evaluation metrics and validation datasets are used to ensure the accuracy and generalization ability of the model.

### 4. Final Simulation and Validation

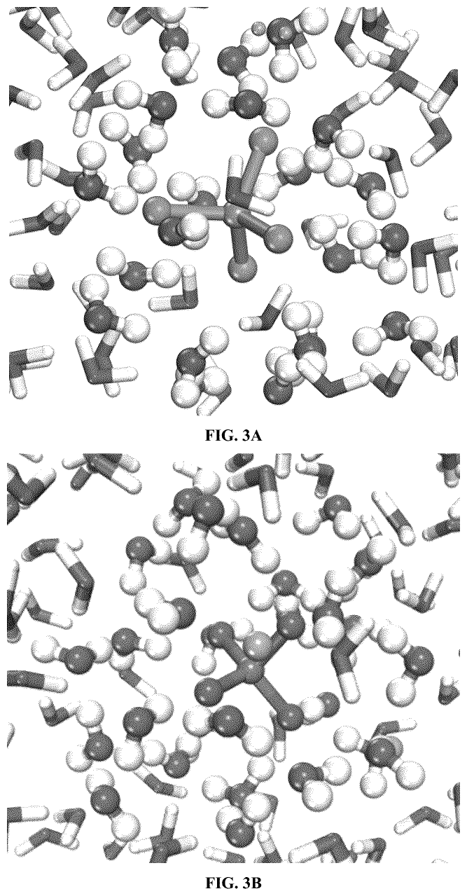

A trained ML force field is applied to the LAMMPS, and a 100 ps ML-accelerated molecular dynamics (i.e. DPMD) simulation is conducted on the DP model, ultimately acquiring the hydration structure of MoS₄²⁻_64H₂O_2Na (FIG. 3A).

The computational costs are as follows.

In terms of pre-optimization of MoS₄²⁻_6H₂O, running continuously for 2 hours on a high-performance computing server with a total of 48 cores at one node results in a computing service fee of approximately 4.8 yuan (0.05 yuan/core hour).

In terms of generation of the MoS₄²⁻_64H₂O_2Na dataset, running continuously for 2 hours on a high-performance computing server with a total of 96 cores at 2 nodes results in a computing service fee of approximately 9.6 yuan (0.05 yuan/core hour), and 20 perturbed structures take a total of 40 hours, resulting in a computing service fee of 192 yuan.

In terms of training of the MoS₄²⁻_64H₂O_2Na model, a round of iteration of the dataset on a 3090Ti graphics card takes 7 days. To ensure the accuracy of the model, 3 rounds of iterations are conducted, which take a total of 504 hours, resulting in a computational service fee of approximately 1,028.16 yuan (2.04 yuan/core hour).

In terms of the DPDM simulation of MoS₄²⁻_64H₂O_2Na, a 100 ps trajectory generated by LAMMPS takes 2 hours on the 3090Ti graphics card, resulting in a computational service fee of approximately 4.08 yuan (2.04 yuan/core hour).

In this way, the hydration structure MoS₄²⁻_64H₂O_2Na is acquired through the systematic 100 ps DPDM simulation, which takes 548 hours and a total of 1,229.04 yuan. In contrast, a 100 ps AIMD simulation requires uninterrupted operation for about 60 days and costs about 70,000 yuan. Therefore, the ML-accelerated anion hydration structure calculation method proposed by the present disclosure improves computational efficiency and reduces computational costs while ensuring computational accuracy.

### Embodiment 2: Tungstate Anion Hydration (WO₄²⁻_64H₂O_2Na)

As shown in FIG. 1, a prediction method for tungstate anion hydration (WO₄²⁻_64H₂O_2Na) is as follows.

### 1. Structure Construction and Optimization

In order to more accurately describe and construct a hydration structure of WO₄²⁻, based on first-principles DFT, the structure of WO₄²⁻_6H₂O is optimized through a def2-TZVP basis set prepared by an ωB97XD functional, thereby generating a stern layer hydration structure of WO₄²⁻.

The anion hydration structure is constructed through Materials Studio software. As shown in FIG. 2B, a WO₄²⁻_64H₂O_2Na model is constructed in a periodic box of 15×15×15 Å. WO₄²⁻ is located at a center of the box, while the remaining 58 H₂O are uniformly and randomly distributed around WO₄²⁻_6H₂O, and Na⁺ is placed at an edge of the box to balance a charge. The constructed anion hydration structure WO₄²⁻_64H₂O_2Na is optimized through VASP.

### 2. Dataset Generation

The optimized WO₄²⁻_64H₂O_2Na structure is perturbed, including changing an atomic coordinate position and a size of the simulation box to generate 20 different perturbed structures. A short-term AIMD simulation is conducted on the optimized structure WO₄²⁻_64H₂O_2Na in an NVT ensemble at 298.15 K, with each configuration spanning 20 frames based on a time step of 0.5 fs.

In this way, a training dataset of the hydration structure WO₄²⁻_64H₂O_2Na is acquired as basic training data for a DeepMD model. It includes 20 perturbed structures, each with 20 frames, totaling 400 frames.

### 3. Model Training and Accelerated Computation

Through DP-GEN and DeePMD kit software package, a ML force fields training is conducted on the training dataset of the hydration structure WO₄²⁻_64H₂O_2Na.

Firstly, a 4×10⁵-step training process is conducted on the training dataset of WO₄²⁻_64H₂O_2Na. 4 independent ML models are established for each training session. These models use the same reference dataset but have different initial weight values.

Then, the trained ML model is applied to a molecular dynamics simulation to explore a new anion hydration structure. The molecular dynamics simulation is conducted by a LAMMPS on the anion hydration structure WO₄²⁻_64H₂O_2Na in the NVT ensemble under five different temperature conditions of 250 K, 280 K, 300 K, 320 K, and 350 K. During the simulation, atomic structures with a force deviation ranging within 0.11-0.30 eV/Å are identified as candidate configurations, and a maximum of 300 candidate configurations are selected.

Finally, a candidate configuration with an energy and atomic force validated through the DFT is merged into the training set for the subsequent iteration to further refine and train the ML model. All iterative processes are automated through the DP-GEN software package until the model converges (i.e., the energy accuracy of the sampled data reaches over 95%) to acquire an accurate DP model. During the validation and testing phase of the model, different evaluation metrics and validation datasets are used to ensure the accuracy and generalization ability of the model.

### 4. Final Simulation and Validation

A trained ML force field is applied to the LAMMPS, and a 100 ps ML-accelerated molecular dynamics (i.e. DPMD) simulation is conducted on the DP model, ultimately acquiring the hydration structure of WO₄²⁻_64H₂O_2Na (FIG. 3B).

The computational costs are as follows.

In terms of pre-optimization of WO₄²⁻_6H₂O, running continuously for 2 hours on a high-performance computing server with a total of 48 cores at one node results in a computing service fee of approximately 4.8 yuan (0.05 yuan/core hour).

In terms of generation of the WO₄²⁻_64H₂O_2Na dataset, running continuously for 2 hours on a high-performance computing server with a total of 96 cores at 2 nodes results in a computing service fee of approximately 9.6 yuan (0.05 yuan/core hour), and 20 perturbed structures take a total of 40 hours, resulting in a computing service fee of 192 yuan.

In terms of training of the WO₄²⁻_64H₂O_2Na model, a round of iteration of the dataset on a 3090Ti graphics card takes 7 days. To ensure the accuracy of the model, 3 rounds of iterations are conducted, which take a total of 504 hours, resulting in a computational service fee of approximately 1,028.16 yuan (2.04 yuan/core hour).

In terms of the DPDM simulation of WO₄²⁻_64H₂O_2Na, a 100 ps trajectory generated by LAMMPS takes 2 hours on the 3090Ti graphics card, resulting in a computational service fee of approximately 4.08 yuan (2.04 yuan/core hour).

In this way, the hydration structure WO₄²⁻_64H₂O_2Na is acquired through the systematic 100 ps DPDM simulation, which takes 548 hours and a total of 1,229.04 yuan. In contrast, a 100 ps AIMD simulation requires uninterrupted operation for about 60 days and costs about 70,000 yuan. Therefore, the ML-accelerated anion hydration structure calculation method proposed by the present disclosure improves computational efficiency and reduces computational costs while ensuring computational accuracy.

In the above embodiments, there are differences in the hydration structures of WO₄²⁻ and MoS₄²⁻ anions, with WO₄²⁻ having a thicker hydration layer and a larger hydration coordination number than MoS₄²⁻. Therefore, compared to MoS₄²⁻, WO₄²⁻ undergoes stronger hydration shielding in the aqueous solution, resulting in selective separation of WO₄²⁻ and MoS₄²⁻ by quaternary ammonium cations during extraction or ion exchange resin adsorption.

### Embodiment 3

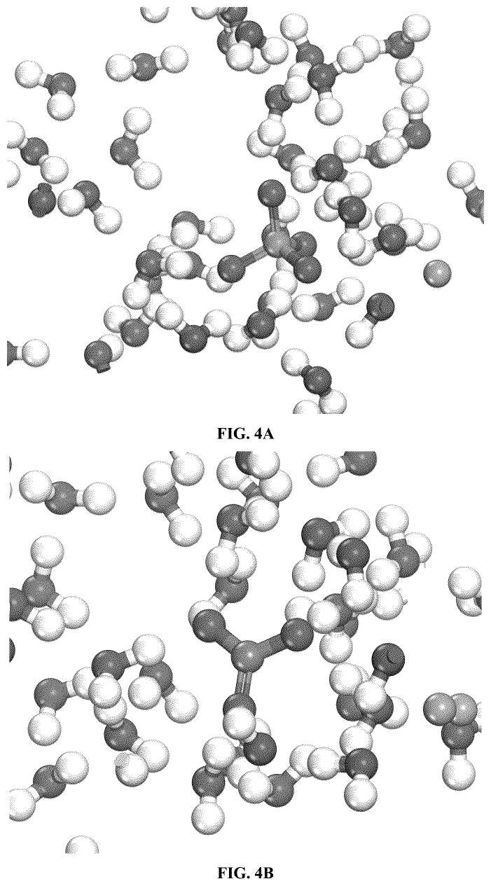

The hydration structures of SO₄²⁻ and CO₃²⁻ are predicted according to the method in Embodiment 1, and the hydration structures of [SO₄(H₂O)₆₄]²⁻ and [CO₃(H₂O)₆₄]²⁻ in the 100 ps DPMD simulation are acquired. As shown in FIGS. 4A-4B, FIG. 4A shows the hydration structure of [SO₄(H₂O)₆₄]²⁻ in the 100 ps DPMD simulation, and FIG. 4B shows the hydration structure of [CO₃(H₂O)₆₄]²⁻ in the 100 ps DPMD simulation.

In this way, the hydration structures of SO₄²⁻_64H₂O_2Na and CO₃²⁻_64H₂O_2Na are acquired through the systematic 100 ps DPDM simulation, each taking approximately 500 hours and approximately 1,120 yuan. In contrast, a 100 ps AIMD simulation requires uninterrupted operation for about 60 days and costs about 70,000 yuan. Therefore, the ML-accelerated anion hydration structure calculation method proposed by the present disclosure improves computational efficiency and reduces computational costs while ensuring computational accuracy.

The above prediction method of the present disclosure can quickly and accurately predict the hydration behaviors of acid radical anions (such as WO₄²⁻, MoS₄²⁻, SO₄²⁻, CO₃²⁻) under different solution conditions, has broad application prospects in complex hydrochemistry, electrochemistry, environmental science, biochemistry, and other fields, and can accelerate related scientific research and technological development processes. For example, the method can be used to simulate and predict the migration and transformation processes of pollutants in water bodies, which can help develop pollution control strategies and environmental remediation plans. The method can help understand the hydration behaviors of acid radical anions in drug molecules, and optimize drug solubility and stability research. In addition, the method can be used to study anion binding sites in biomacromolecule (such as proteins and nucleic acids), and understand their functions in biological systems.

The above embodiments describe the preferred implementations of the present disclosure in detail. However, the present disclosure is not limited to specific details of the above implementations. A plurality of simple variations can be made to the technical solutions of the present disclosure within the scope of the technical idea of the present disclosure, and all of these simple variations fall within the protection scope of the present disclosure.

In addition, it should be noted that various specific technical features described in the above specific embodiments can be combined in any suitable manner, provided that there is no contradiction. To avoid unnecessary repetition, various possible combination modes of the present disclosure are not described separately.

In addition, different implementations of the present disclosure can also be combined arbitrarily. The combinations should also be regarded as the content disclosed in the present disclosure, provided that they do not violate the ideas of the present disclosure.

What is claimed is:

1. A machine learning (ML)-accelerated first-principles prediction method for a hydration structure of an acid radical anion, comprising the following steps:
S1: constructing and optimizing an anion hydration structure M_mH₂O;
S2: perturbing an optimized anion hydration structure to generate a training dataset;
S3: conducting a ML force field training on the training dataset to establish ML models;
S4: conducting a molecular dynamics simulation on the ML models, and identifying atomic structures with a force deviation within a preset range as candidate configurations;
S5: merging a validated candidate configuration into a training set for a subsequent iteration to further refine and train the ML models until the ML models converge, thereby generating an accurate deep potential (DP) model; and
S6: conducting a ML-accelerated molecular dynamics simulation on the accurate DP model to ultimately acquire the hydration structure of the acid radical anion;
wherein in the step S1, a method for constructing the anion hydration structure M_mH₂O comprises: creating a periodic simulation box, placing an acid radical anion M at a center of the periodic simulation box, uniformly and randomly distributing m H₂O around the acid radical anion M, and placing an alkali metal cation at an edge of the periodic simulation box to balance a charge, thereby completing a construction of an initial anion hydration structure M_mH₂O, wherein m is above 40;
before constructing the anion hydration structure M_mH₂O, the ML-accelerated first-principles prediction method further comprises a pre-optimization, comprising: preparing a def2-TZVP, def2-SVP, def2-QZVP, def2-TZVPP, cc-pVDZ-PP, or aug-cc-pVDZ-PP basis set through an ωB97XD, B3LYP, or PBE functional; and pre-optimizing a simple hydration structure M_nH₂O formed by n H₂O in a sternest layer around the acid radical anion M to acquire a stern layer hydration structure of the acid radical anion M, wherein n is not more than 10; uniformly and randomly distributing a remaining (m-n) H₂O around the initial anion hydration structure M_nH₂O, thereby completing the construction of the initial anion hydration structure M_mH₂O; and optimizing a constructed anion hydration structure through a Vienna ab initio simulation package; and
in the step S2, the perturbing comprises: changing an atomic coordinate position and a size of the periodic simulation box to generate A different perturbed structures, each of the A different perturbed structures being in a canonical ensemble (NVT ensemble) at 298.15 K; and recording B frames for each configuration at a time step of 0.5-1.5 fs, and conducting a short-term ab initio molecular dynamics (AIMD) simulation to generate a training dataset of A×B frames as a basic training data for a DeepMD model, wherein A is 15-30; and B is 15-25.

2. The ML-accelerated first-principles prediction method according to claim 1, wherein the time step is 0.5 fs; the A is 20; and the B is 20.

3. The ML-accelerated first-principles prediction method according to claim 1, wherein in the step S3, the ML force field training comprises 2×10⁵ to 8×10⁵ steps; 2-5 independent ML models are established each time; and each of the 2-5 independent ML models has a different initial weight value.

4. The ML-accelerated first-principles prediction method according to claim 1, wherein in the step S4, the conducting the molecular dynamics simulation comprises: conducting, by a large-scale atomistic/molecular massively parallel simulator (LAMMPS), the molecular dynamics simulation on the optimized anion hydration structure in the NVT ensemble under at least five different temperature conditions; and identifying, during the molecular dynamics simulation, the atomic structures with the force deviation ranging within 0.11-0.30 eV/Å as the candidate configurations, wherein a maximum of 300 candidate configurations are selected.

5. The ML-accelerated first-principles prediction method according to claim 4, wherein the molecular dynamics simulation is conducted by the LAMMPS on the optimized anion hydration structure in the NVT ensemble under five different temperature conditions of 250 K, 280 K, 300 K, 320 K, and 350 K.

6. The ML-accelerated first-principles prediction method according to claim 1, wherein the step S5 comprises: merging the validated candidate configuration with an energy and an atomic force validated through a density functional theory (DFT) into the training set for the subsequent iteration.

7. The ML-accelerated first-principles prediction method according to claim 1,wherein the step S6 comprises: applying a trained ML force field to an LAMMPS, and conducting a 50-1,000 ps ML-accelerated deep potential molecular dynamics (DPMD) simulation on the accurate DP model to ultimately acquire the hydration structure of the acid radical anion.

8. The ML-accelerated first-principles prediction method according to claim 1, wherein the acid radical anion is WO₄²⁻, MoS₄²⁻, SO₄²⁻, or CO₃²⁻.

9. The ML-accelerated first-principles prediction method according to claim 2, wherein the step S6 comprises: applying a trained ML force field to an LAMMPS, and conducting a 50-1,000 ps ML-accelerated deep potential molecular dynamics (DPMD) simulation on the accurate DP model to ultimately acquire the hydration structure of the acid radical anion.

10. The ML-accelerated first-principles prediction method according to claim 3, wherein the step S6 comprises: applying a trained ML force field to an LAMMPS, and conducting a 50-1,000 ps ML-accelerated deep potential molecular dynamics (DPMD) simulation on the accurate DP model to ultimately acquire the hydration structure of the acid radical anion.

11. The ML-accelerated first-principles prediction method according to claim 4, wherein the step S6 comprises: applying a trained ML force field to an LAMMPS, and conducting a 50-1,000 ps ML-accelerated deep potential molecular dynamics (DPMD) simulation on the accurate DP model to ultimately acquire the hydration structure of the acid radical anion.

12. The ML-accelerated first-principles prediction method according to claim 5, wherein the step S6 comprises: applying a trained ML force field to an LAMMPS, and conducting a 50-1,000 ps ML-accelerated deep potential molecular dynamics (DPMD) simulation on the accurate DP model to ultimately acquire the hydration structure of the acid radical anion.

13. The ML-accelerated first-principles prediction method according to claim 6, wherein the step S6 comprises: applying a trained ML force field to an LAMMPS, and conducting a 50-1,000 ps ML-accelerated deep potential molecular dynamics (DPMD) simulation on the accurate DP model to ultimately acquire the hydration structure of the acid radical anion.

14. The ML-accelerated first-principles prediction method according to claim 2, wherein the acid radical anion is WO₄²⁻, MoS₄²⁻, SO₄²⁻, or CO₃²⁻.

15. The ML-accelerated first-principles prediction method according to claim 3, wherein the acid radical anion is WO₄²⁻, MoS₄²⁻, SO₄²⁻, or CO₃²⁻.

16. The ML-accelerated first-principles prediction method according to claim 4, wherein the acid radical anion is WO₄²⁻, MoS₄²⁻, SO₄²⁻, or CO₃²⁻.

17. The ML-accelerated first-principles prediction method according to claim 5, wherein the acid radical anion is WO₄²⁻, MoS₄²⁻, SO₄²⁻, or CO₃²⁻.

18. The ML-accelerated first-principles prediction method according to claim 6, wherein the acid radical anion is WO₄²⁻, MoS₄²⁻, SO₄²⁻; or CO₃²⁻.
